(19) United States
(12) Patent Application Publication
(10) Pub. No.: US 2026/0007905 A1
(43) Pub. Date: Jan. 8, 2026

(54) MEDICAL LASER DEVICE

(71) Applicant: Dong Hyuk SONG, Seoul (KR)

(72) Inventor: Dong Hyuk SONG, Seoul (KR)

(21) Appl. No.: 19/124,215

(22) PCT Filed: Oct. 24, 2023
(86) PCT No.: PCT/KR2023/016567

§ 371 Date: Apr. 24, 2025

(30) Foreign Application Priority Data
Oct. 31, 2022 (KR) 20-2022-0002536

(51) Int. Cl. A61N 5/067; A61N 5/06

(52) U.S. Cl. CPC A61N 5/067; A61N 2005/0644; A61N 2005/0667

(57) ABSTRACT

The present invention proposes a medical laser device. A medical laser device according to an embodiment of the present invention includes a handpiece for emitting a laser frontward, the laser being received from at least one laser module for generating the laser, wherein the handpiece is configured to be grippable and includes a body having a receiving space therein and a protective cap disposed at the end of the body and formed to have a cylindrical shape such that a laser emitted from the body can pass therethrough. The protective cap is coated by a material for absorbing a wavelength of a laser emitted from the handpiece or has a filter which prevents the laser from passing therethrough and is disposed on an inner surface and/or an outer surface thereof, and is used to be replaceable according to a wavelength of a laser emitted from the handpiece.

### TECHNICAL FIELD

The present invention relates to a medical laser device.

### BACKGROUND ART

Lasers have been used in various fields such as industrial, medical, and military fields. In particular, medical laser devices have been used in ophthalmic clinics, dental clinics, surgical operations, dermatologic clinics, and the like. For example, the medical laser devices may be used to treat lesions such as skin diseases or vascular diseases that occur on the skin by emitting lasers to the lesions. In addition, the medical laser devices have been used to perform various types of surgeries on living tissues in the skin beauty treatment field.

In general, since the medical laser devices require precise work, the medical laser devices are manufactured in the form of handpieces so that users may grip the devices with their hands to use the devices. The medical laser devices have been used for various purposes such as hair loss prevention or hair growth promotion, skin peeling, skin regeneration, whitening, wrinkle or spot removal, and freckle removal. Except that the medical laser devices use lasers, there are differences in devices and techniques according to the purpose of use, and even for the same purpose, there are differences in conditions for emitting lasers or devices used.

In general, when a laser is emitted from a laser device to the skin of a procedure recipient so as to perform a treatment, a practitioner wears protective goggles for protecting eyes from the laser. Such protective goggles have filters, which have characteristics that block a wavelength of the laser while transmitting visible light, and are disposed in front of the eyes.

In addition, when the wavelength of the laser is changed, protective goggles to which filters capable of responding to multi-wavelength lasers are attached are worn, or protective goggles to which dedicated filters for respective wavelengths are attached are prepared, so that the practitioner may wear the protective goggles selectively according to a selected wavelength.

However, it is difficult for protective goggles capable of blocking all multi-wavelength lasers to obtain necessary attenuation for each wavelength, and the protective goggles have less visibility. Even when a plurality of protective goggles to which dedicated filters for respective wavelengths are attached are prepared, it is inconvenient to put on and take off the protective goggles, and the protective goggles still have less visibility.

Accordingly, the protective goggles alone are insufficient to block the laser, and a device for additionally blocking the laser to reduce eye fatigue of the user and prevent impaired vision is required.

### DISCLOSURE

### Technical Problem

One object of the present invention is to provide a medical laser device capable of protecting eyes of a practitioner, and ensuring stability of a procedure.

However, technical objects to be achieved by the present embodiment are not limited to the technical objects described above, and other technical objects may exist.

### Technical Solution

As a solution for achieving the technical objects described above, according to one embodiment of the present invention, a medical laser device includes: a handpiece for emitting a laser frontward, in which the laser is received from at least one laser module, wherein the handpiece includes: a body configured to be grippable, and having a receiving space therein; and a protective cap located at an end of the body, and having a cylindrical shape such that the laser emitted from the body is able to pass through the protective cap. In this case, the protective cap is coated with a material for absorbing a wavelength of the laser emitted from the handpiece or has a filter configured to prevent the laser from being transmitted through the filter and located on an inner surface and/or an outer surface of the protective cap, and is used to be replaceable according to the wavelength of the laser emitted from the handpiece.

In addition, according to one embodiment of the present invention, a protective cap is coated with a material for absorbing a wavelength of a laser emitted from a handpiece or has a filter configured to prevent the laser from being transmitted through the filter and located on an inner surface and/or an outer surface of the protective cap, and is used to be replaceable according to the wavelength of the laser emitted from the handpiece.

### Advantageous Effects

According to the solution of the present invention described above, only a protective cap located at an end of a handpiece may be replaced according to a wavelength of a laser emitted from the handpiece, so that eyes of a practitioner can be protected, and stability of a procedure can be ensured.

### DESCRIPTION OF DRAWINGS

FIG. 1 is a view showing a medical laser device according to a first embodiment of the present invention.

FIG. 2 is a perspective view showing a guide tip according to one embodiment of the present invention.

FIG. 3 is an enlarged view showing a portion A of FIG. 1.

FIG. 4 is a perspective view showing various types of guide tips according to the present invention.

FIG. 5 is a view showing a medical laser device according to a second embodiment of the present invention.

FIG. 6 is an enlarged view showing a portion B of FIG. 5.

FIG. 7 is a sectional view showing a protective cap according to another embodiment of the present invention.

### BEST MODE

Hereinafter, an embodiment of the present disclosure will be described in detail with reference to the accompanying drawings so as to be easily practiced by a person having ordinary skill in the art to which the present disclosure pertains. However, the present disclosure may be implemented in various different forms without being limited to the embodiment described herein. In addition, in order to clearly describe the present disclosure, in the drawings, parts that are not related to the description are omitted, and like parts are given like reference numerals throughout the present disclosure.

Throughout the present disclosure, when some part is described as being “connected” to another part, this includes not only a case where the part is “directly connected” to the other part, but also a case where the part is “electrically connected” to the other part with another device interposed therebetween.

Throughout the present disclosure, when some member is described as being located “on” another member, this includes not only a case where the member makes contact with the other member, but also a case where another member exists between the two members.

Throughout the present disclosure, when some part is described as “including” some component, unless explicitly described to the contrary, it means that another component may be further included but not excluded. The terms that express degrees, such as “about” and “substantially”, used throughout the present disclosure have meanings that are at a numerical value or close to the numerical value when unique manufacturing and material tolerances are presented in a stated meaning, and are used to prevent unconscientious infringers from unfairly exploiting the disclosure in which an exact or absolute value is stated in order to help understanding of the present disclosure. Throughout the present disclosure, “step that . . . ” or “step of . . . ” do not mean “step for . . . ”.

Hereinafter, exemplary embodiments of the present invention will be described in detail with reference to the accompanying drawings and the contents described below. However, the present invention is not limited to the embodiments described herein, and may be embodied in other forms. Throughout the present disclosure, like reference numerals refer to like elements throughout the present disclosure.

The present invention relates to a medical laser device 10.

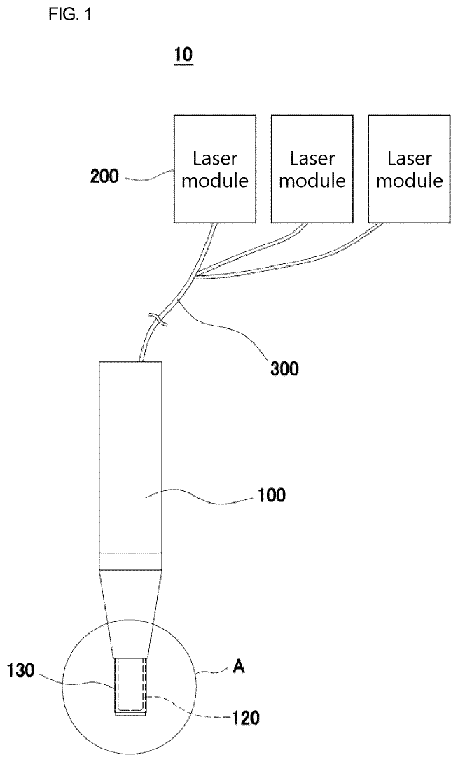

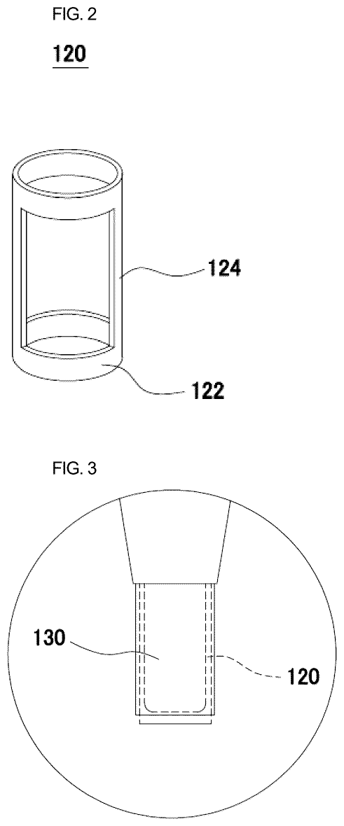

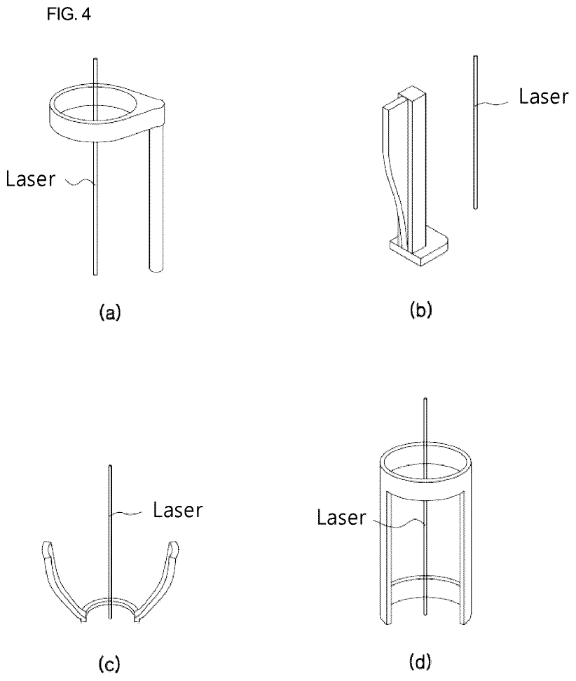

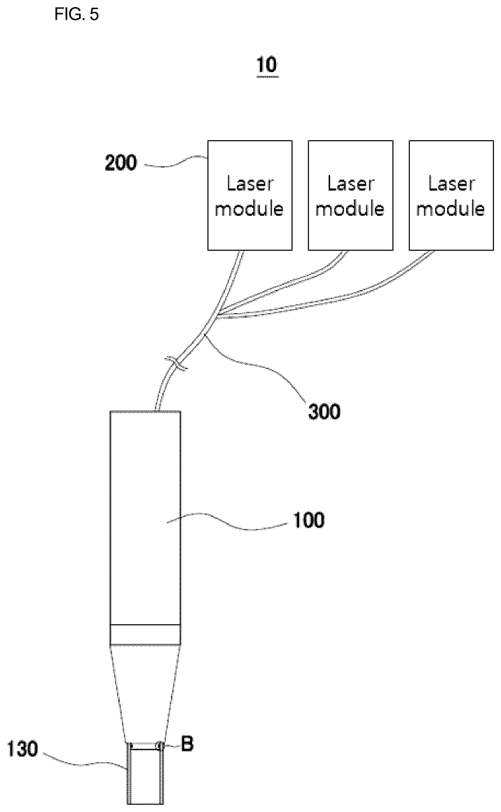

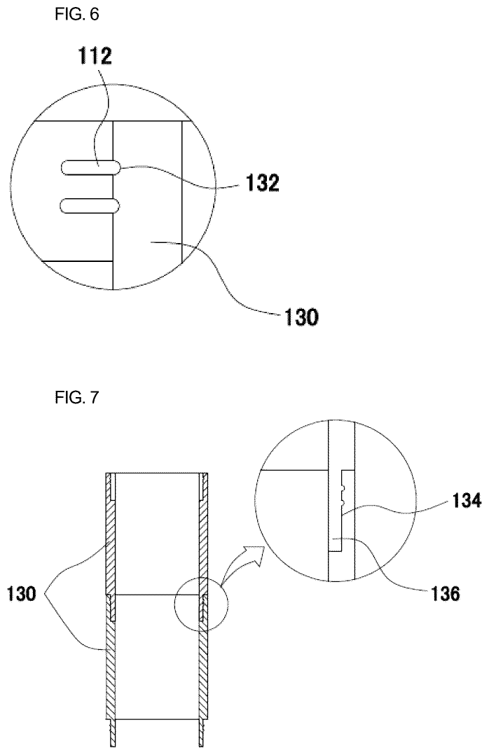

FIG. 1 is a view showing a medical laser device according to a first embodiment of the present invention, FIG. 2 is a perspective view showing a guide tip according to one embodiment of the present invention, FIG. 3 is an enlarged view showing a portion A of FIG. 1, FIG. 4 is a perspective view showing various types of guide tips according to the present invention, FIG. 5 is a view showing a medical laser device according to a second embodiment of the present invention, FIG. 6 is an enlarged view showing a portion B of FIG. 5, and FIG. 7 is a sectional view showing a protective cap according to another embodiment of the present invention.

Hereinafter, a medical laser device 10 according to a first embodiment of the present invention (hereinafter referred to as a “laser device 10”) will be described with reference to FIGS. 1 to 3.

The laser device 10 may include at least one laser module 200 for generating a laser, a cable 300 for transmitting the laser of the laser module 200, and a handpiece 100 connected to the cable 300 and configured to emit the laser frontward.

The laser module 200 may generate a laser having a predetermined wavelength by applying power. In addition, as shown in FIG. 1, a plurality of laser modules 200 may be provided, and the laser modules 200 may generate lasers having different wavelengths. In this case, the wavelength of the laser may be one of 1064 nm, 1320 nm, 532 nm, 535 nm, 585 nm, and 595 nm, but is not limited thereto.

The cable 300 may serve to transmit the laser of the laser module 200 to the handpiece 100, and when a plurality of laser modules 200 are provided, one selected laser module 200 may be connected to the handpiece 100.

The handpiece 100 may be a device for emitting a laser to the skin of a procedure recipient, and may include a body 110 and a protective cap 130 located at an end of the body 110.

The body 110 may be a component gripped by a practitioner, and may accommodate various types of control components, such as a PCB, for driving the handpiece 100. For example, the body 110 may have a columnar shape in which a sectional shape is a circular shape or a polygonal shape, but is not limited thereto.

In addition, the body 110 may include a control unit (not shown) capable of selecting power and a laser wavelength of the handpiece 100. For example, the practitioner may select a laser module 200 for generating a desired wavelength among the laser modules 200 through the control unit.

The protective cap 130 may be located at the end of the body 110, and may have a cylindrical shape such that the laser emitted from the body 110 may pass through the protective cap 130, but the shape of the protective cap 130 is not limited thereto.

In addition, the protective cap 130 may be coated with a material for absorbing a wavelength of the laser emitted from the 5 handpiece 100 or may have a filter configured to prevent the laser from being transmitted through the filter and located on an inner surface and/or an outer surface of the protective cap 130, so that leakage of the laser to an outside may be minimized. Since the material for absorbing the wavelength of the laser and the filter described above are technologies that are generally known, detailed descriptions thereof will be omitted.

In addition, the practitioner may replace and use the protective cap 130 according to the wavelength of the laser emitted from the handpiece 100.

In addition, the handpiece 100 may further include a guide beam emission unit (not shown) for emitting a beam to the same position to which the laser is emitted. In addition, the guide beam emission unit may emit a beam transmitted through the protective cap 130 so as to be recognized from the outside, so that the practitioner may accurately recognize the position to which the laser is emitted.

Referring to FIG. 1, the handpiece 100 may include a guide tip 120 coupled to the end of the body 110, and making contact with a procedure region to guide the laser.

The guide tip 120 may serve to prevent the body 110 from making direct contact with the skin so as to reduce a contact area with the skin so that a skin treatment region may be sufficiently ensured, and may serve to maintain a constant distance between the body 110 and the skin so as to ensure a constant treatment level.

For example, referring to FIG. 2, the guide tip 120 may include a ring part 122 having a ring shape, and at least one support part 124 extending from an upper portion of the ring part 122. In addition, the guide tip 120 may be coupled to the body 110 by interference fit, screw coupling, latched coupling, and the like, but is not limited thereto.

Referring to FIG. 3, the protective cap 130 may be coupled to the guide tip 120 or the end of the body 110 while surrounding the guide tip 120. However, the protective cap 130 is not limited thereto, and the protective cap 130 may also be located inside the guide tip 120.

The protective cap 130 may be coupled to the guide tip 120 by interference fit, screw coupling, latched coupling, and the like, but is not limited thereto.

In addition, an end of the protective cap 130 may be located higher than an end of the guide tip 120. In other words, when the protective cap 130 is coupled to the body 110, and the end of the guide tip 120 makes contact with an emission target part so that the laser may be emitted to the emission target part, the end of the protective cap 130 may be spaced apart from the emission target part by a predetermined distance without making contact with the emission target part so as to form a space, so that the practitioner may check whether the laser is emitted through the formed space.

Hereinafter, various types of guide tips 120 will be described with reference to FIG. 4.

Referring to FIG. 4(a), the guide tip 120 may include a ring-shaped member coupled to the body 100, and a bar support part coupled to a bottom surface of the ring-shaped member and having a bar shape. In other words, the ring-shaped member of the guide tip 120 may be coupled to the body 100, and an end of the bar support part may make contact with the emission target part. Referring to FIG. 4(b), an overall shape of the guide tip 120 may be a bar shape, and a portion making contact with the emission target part may have a predetermined area. In this case, the guide tip 120 may be located on one side of a portion to which the laser is emitted, so that the laser may be emitted to a portion spaced apart from the end of the guide tip 120 by a predetermined distance.

Referring to FIG. 4(c), the guide tip 120 may be configured such that a portion making contact with the emission target part has an arc shape and extends upward and outward from both end portions of an arc-shaped member, so that a support member may be fixed to the body 100. In this case, the laser may be emitted to a center point of the arc-shaped member, and the emission of the laser may be clearly recognized through an open portion.

Referring to FIG. 4(d), the guide tip 120 may be configured such that a portion coupled to the body 100 has a cylindrical shape, a portion making contact with the emission target part has an arc shape, and both end portions of a bar-shaped member may be connected to a cylindrical member and an arc-shaped member, respectively. In this case, the laser may be emitted to the center point of the arc-shaped member, and the emission of the laser may be clearly recognized through an open portion.

Although the guide tips 120 according to various embodiments have been described with reference to FIG. 4, the guide tip 120 may have various shapes without being limited to the contents described above, and the shape of the protective cap 130 may be changed according to the shape of the guide tip 120.

Hereinafter, a medical laser device 10 according to a second embodiment of the present invention will be described with reference to FIGS. 5 to 7.

As shown in FIG. 5, unlike the first embodiment, the laser device 10 may be configured such that the protective cap 130 may be directly connected to the body 110 without being connected through the guide tip 120. In this case, the protective cap 130 may serve as the guide tip 120. In other words, the protective cap 130 may be directly coupled to the end of the handpiece 100.

Referring to FIG. 6, the protective cap 130 may include coupling grooves 132 formed on the inner surface of the protective cap 130 while being spaced apart from each other by a predetermined interval in a length direction, and the end of the body 110 may include protrusion parts 112 formed at positions corresponding to the coupling grooves 132. However, the protective cap 130 is not limited thereto, and the protrusion part 112 may be formed on the protective cap 130, and the coupling groove 132 may be formed on the end of the body 110.

In detail, when the protective cap 130 is coupled to the body 110, an upper opening part may be inserted into a portion in which the coupling groove 132 is formed at the end of the body 110 so as to be pressed, so that the protrusion part 112 may be inserted into the coupling groove 132 so as to be firmly fixed. Conversely, when the protective cap 130 is separated from the body 110, in a case where the protective cap 130 is pulled in an opposite direction to a position in which the body 110 is located, the protrusion part 112 may be detached from the coupling groove so that the protective cap 130 may be separated from the body 110.

The protective cap 130 and the body 110 may be coupled to each other not only by the latched coupling described above, but also by the screw coupling. For example, a screw thread may be formed on the inner surface of the protective cap 130, and a screw thread may be formed on an outer peripheral surface of the end of the body 110, so that the protective cap 130 may be rotated to allow the screw threads to be coupled to each other. In this case, a distance between the skin of the procedure recipient and the body 110 may be adjusted by increasing a length of a portion in which the screw thread is formed, and adjusting the number of rotations of the protective cap 130.

In addition, the protective cap 130 may include a laser checking hole (not shown) formed by perforating a portion of a lower circumferential part of the protective cap 130. The practitioner may check whether the laser is emitted through the laser checking hole.

In addition, referring to FIG. 7, according to the present invention, a distance between the skin of the procedure recipient and the body 110 may be adjusted by connecting at least two protective caps 130 to each other.

To this end, the protective cap 130 may include a step part 134 formed on the inner surface of the protective cap 130 and stepped by a predetermined depth in an outward direction, and a step coupling part 136 formed on the outer surface of the protective cap 130 and stepped by a predetermined depth in an inward direction. In addition, a length of the protective cap 130 may be extended by coupling the step part 134 of the protective cap 130 to a step coupling part 136 of another protective cap 130.

The above description of the present invention is for illustrative purposes, and it will be understood by a person having skill in the art to which the present invention pertains that the present invention can be easily modified in other specific forms without changing the technical idea or essential characteristics of the present invention. Therefore, the embodiments described above are illustrative in all aspects, and are not to be construed as limiting. For example, each component described in a single form may be implemented in a distributed manner, and similarly, components described as being distributed may be implemented in a combined form.

The scope of the present invention is defined by the claims described below rather than the detailed description, and the scope of the present invention is to be interpreted as encompassing all changes or modifications derived from the meaning and scope of the claims and equivalent concepts thereof.

### DESCRIPTION OF REFERENCE NUMERALS

10: Medical laser device
100: Handpiece
110: Body 112: Protrusion part
120: Guide tip 122: Ring part
124: Support part
130: Protective cap 132: Coupling groove
134: Step part 136: Step coupling part
200: Laser module
300: Cable

1. A medical laser device comprising:
a handpiece for emitting a laser frontward, in which the laser is received from at least one laser module,
wherein the handpiece includes:
a body configured to be grippable, and having a receiving space therein; and
a protective cap located at an end of the body, and having a cylindrical shape such that the laser emitted from the body is able to pass through the protective cap, and
the protective cap is coated with a material for absorbing a wavelength of the laser emitted from the handpiece or has a filter configured to prevent the laser from being transmitted through the filter and located on an inner surface and/or an outer surface of the protective cap, and is used to be replaceable according to the wavelength of the laser emitted from the handpiece.

2. The medical laser device of claim 1, further comprising:
one or more laser modules; and
a cable for transmitting lasers of the laser modules to the handpiece,
wherein the laser modules transmit the lasers having different wavelengths to the handpiece.

3. The medical laser device of claim 1, wherein the handpiece includes:
a guide tip coupled to the end of the body, and making contact with a procedure region to guide the laser.

4. The medical laser device of claim 3, wherein the end of the protective cap is located higher than an end of the guide tip.

5. The medical laser device of claim 1, further comprising:
a guide beam emission unit for emitting a beam, in which the beam is emitted to a position of an emission target part to which the laser is to be emitted, and the beam is transmitted through the protective cap so as to be recognizable from an outside.

6. The medical laser device of claim 1, wherein the protective cap is coupled to an end of the handpiece.

7. The medical laser device of claim 6, wherein the protective cap includes coupling grooves formed on the inner surface of the protective cap while being spaced apart from each other by a predetermined interval in a length direction, and
the end of the body includes protrusion parts formed at positions corresponding to the coupling grooves.

8. The medical laser device of claim 6, wherein the protective cap includes a step part formed on the inner surface of the protective cap and stepped by a predetermined depth in an outward direction, and a step coupling part formed on the outer surface of the protective cap and stepped by a predetermined depth in an inward direction, and
the step part of the protective cap is coupled to a step coupling part of another protective cap.

9. A protective cap mounted on a medical laser device, wherein the protective cap is coated with a material for absorbing a wavelength of a laser emitted from a handpiece or has a filter configured to prevent the laser from being transmitted through the filter and located on an inner surface and/or an outer surface of the protective cap, and is used to be replaceable according to the wavelength of the laser emitted from the handpiece.

10. The protective cap of claim 9, wherein the protective cap has an end located higher than an end of a guide tip making contact with a procedure region.

11. The protective cap of claim 9, wherein the protective cap is coupled to an end of the handpiece, or a guide tip.

12. The protective cap of claim 9, wherein the protective cap includes coupling grooves formed on the inner surface of the protective cap while being spaced apart from each other by a predetermined interval in a length direction, and
an end of the handpiece includes protrusion parts formed at positions corresponding to the coupling grooves.

13. The protective cap of claim 9, wherein the protective cap includes a step part formed on the inner surface of the protective cap and stepped by a predetermined depth in an outward direction, and a step coupling part formed on the outer surface of the protective cap and stepped by a predetermined depth in an inward direction, and
the step part of the protective cap is coupled to a step coupling part of another protective cap.
